(19) United States
(12) Patent Application Publication
(10) Pub. No.: US 2020/0043574 A1
(43) Pub. Date: Feb. 6, 2020

(54) Network-Based Systems for Analysis-Based Authentication and Monetization

(71) Applicant: Oak Analytics Inc., Agoura Hills, CA (US)

(72) Inventors: Deepak Mehrotra, Thousand Oaks, CA (US); Steve K. Chen, Pacific Palisades, CA (US)

(73) Assignee: Oak Analytics Inc., Agoura Hills, CA (US)

(21) Appl. No.: 16/583,038

(22) Filed: Sep. 25, 2019

Related U.S. Application Data
(63) Continuation-in-part of application No. 16/362,597, filed on Mar. 22, 2019.
(60) Provisional application No. 62/646,693, filed on Mar. 22, 2018.

(51) Int. Cl. G16C 20/20; H04L 29/08; G06Q 30/04; G06Q 30/00

(52) U.S. Cl. CPC G16C 20/20; H04L 67/1097; G01N 21/65; G06Q 30/04; G06Q 30/0185; H04L 67/04

(57) ABSTRACT

Verification systems for testing food products or other samples may include a mobile analytical device, a mobile accessory device such as a smart phone, and a remote, e.g., cloud-based, computing system. The mobile analytical device is adapted to generate a sensor output that is indicative of a molecular composition of the sample. The mobile accessory device may be adapted to receive the sensor output from the mobile analytical device. The remote computing system may be adapted to analyze analytical data using artificial intelligence (AI) and/or machine learning (M-L) to make an authentication determination of the sensor output relative to a predefined product database. The mobile accessory device may be adapted to upload the sensor output to the remote computing system by a communication network, and the remote computing system may be adapted to download the authentication determination to the mobile accessory device by the same communication network.

### CROSS-REFERENCE TO RELATED APPLICATIONS

This application is a continuation-in-part under 35 U.S.C. § 120 of patent application U.S. Ser. No. 16/362,597, “Network-Based. Verification Systems and Methods”, filed Mar. 22, 2019 and currently pending, which claims priority under 35 U.S.C. § 119 (e) to provisional patent application U.S. Ser. No. 62/646,693, “Click and Bill”, filed Mar. 22, 2018 and now expired, the contents of each of which are incorporated herein by reference.

### FIELD OF THE INVENTION

The present invention relates to systems designed to verify the chemical composition or similar characteristic of a product, material, or other sample of interest. In some cases such systems may analyze food samples using Raman spectroscopy or other spectroscopic techniques, or alternative measurement techniques. Aspects of the invention also relate to an automatic monetization procedure for such systems. The invention also pertains to related methods, systems, and articles.

### BACKGROUND OF THE INVENTION

Recent reports point to the existence of a global food safety crisis due to widespread counterfeiting and adulteration of food products. For example, a Global Food Safety Initiative Report estimated that food fraud costs the global food industry $30-$40 billion annually. A press release by Europol (Apr. 25, 2017) reported that in the first four months of 2017, Europol and Interpol seized 230 million Euros worth of fake food and beverages. A 2015-2016 annual report of the FSSAI (Indian counterpart to the U.S. Food & Drug Administration) reported that in India during 2015-2016, one out of five food samples analyzed was adulterated.

Sophisticated counterfeiters are quick to imitate successful products, e.g. as illustrated by the widespread and aggressive counterfeiting of Moutai brand liquor in China. Attempts to stop the counterfeiting by the use of various security features on the product packaging, including QR codes, holograms, cap security seals, and RFID tracking, have all failed. Counterfeiters may require only 9-12 months to duplicate such packaging features. Thus, securing the packaging of a product in ways such as this does not verify or guarantee that the contents are authentic.

From a marketing standpoint, food testing is a large and expanding market sector, estimated to grow from $12 billion in 2016 (of which rapid testing constitutes $1.2 billion) to $18.5 billion in 2022 (of Which rapid testing will constitute $3.7 billion).

### SUMMARY OF THE INVENTION

A need exists in the industry for new, more effective verification systems for food products or other samples, and for simplifying and streamlining transaction procedures, including billing procedures, for such systems. The application of increasingly effective technologies at different packaging levels can help build an efficient anti-counterfeiting strategy. Relative to product packaging, such as tamper-evident outer pack closure systems including seals, glued flaps, and perforated cartons, authentication technologies such as overt and covert features can provide increased protection. Relative to authentication technologies, traceability technologies such as unique pack identifiers (serial number) combined with online checking systems (end-to-end electronic pedigree) can provide still more protection. Relative to traceability technologies, molecular analysis can provide even more protection.

We have developed a new family of verification systems and methods that can employ molecular analysis—in some cases, Raman molecular analysis—with devices that can analyze the product of interest or sample in situ, through the product's outer bottle or container without opening or breaking the seal of the container. Raman spectroscopy is the spectral analysis of light scattered from the sample, the scattered light providing a unique molecular signature based on the (molecular) structure and composition of the sample material. When employed in the disclosed systems, Raman spectroscopy can allow for instant, nearly instantaneous, or at least extremely rapid product verification of the contents of the container.

The disclosed systems may combine Raman spectroscopy, or other analytical detection techniques, with advanced computer technologies such as artificial intelligence (AI), machine learning (M-L), and/or Big Data. Machine learning is an application of AI that provides systems with the ability to automatically learn and improve from experience without being explicitly programmed by a human. Big Data refers to the process of examining large and varied data sets, or big data, to uncover or ascertain information including hidden patterns, unknown correlations, such as changes in fluorescence patterns of certain oils by addition of different substitutions/adulterants.

The system architecture preferably employs portable, handheld, or compact devices for some of the system functionality, and remote (e.g. cloud-based) computer(s) for at least some of the advanced computer technologies. If desired, computational tasks such as those associated with AI or can be performed on the remote computer(s), while the raw data to be analyzed, and the output verification result (e.g. pass/fail) calculated by the remote computer, can be transmitted rapidly to a mobile accessory device such as the user's smart phone. In alternative embodiments, the authentication or verification result can be calculated by the mobile accessory device itself after receiving one or more data files from the remote computer. The disclosed systems and methods are also preferably configured to automatically and rapidly invoice or bill the user, or a designated person or entity, for the analysis or analyses performed.

We therefore disclose herein, among other things, verification systems for testing a product or other sample, comprising a mobile analytical device, a mobile accessory device, and a remote computing system. The mobile analytical device may be adapted to generate a sensor output that is characteristic of the molecular composition of the sample. The mobile accessory device may be adapted to receive the sensor output from the mobile analytical device. The remote computing system may be adapted to analyze analytical data using AI and/or M-L to make an authentication determination of the sensor output relative to a predefined product database. The mobile accessory device may be adapted to upload the sensor output to the remote computing system by a communication network, and the remote computing system may be adapted to download the authentication determination to the mobile accessory device by the communication network. The authentication determination may be or include a verification result whose value is indicative of the likelihood that the measured sample is authentic, based on the analysis of the sample's properties as measured by the analytical device.

In some cases, the mobile accessory device may be or include a smart phone. In some eases, the mobile analytical device may be or include a compact spectrometer, and the sensor output may be or include a Raman spectrum. In some cases, the remote computing system may be a cloud-based computing system. In some cases, the mobile analytical device may include a button which, when activated by a user, causes the mobile analytical device to analyze the sample, generate the sensor output, and transmit the sensor output to the mobile accessory device. The activation of the button by the user may further cause the mobile accessory device to upload the sensor output to the remote computing system, and cause the remote computing system to make the authentication determination and download the authentication determination, such as a verification result, to the mobile accessory device. The activation of the button by the user may further cause the remote computing system to calculate a billing output for the sample analysis performed by the mobile analytical device. Such billing output may be calculated as a function of one, some, or all of (a) whether the authentication determination is positive or negative, or (b) an identity of the sample, or (c) an identity or group of the user.

In some cases, the remote computing system may include a System Critic module, a Decider module, a Learner module, a Monetizer (Monetization) module, spectral database(s), calibration database(s), and/or user database(s).

We also disclose verification systems for testing a sample, comprising a mobile analytical device, a mobile accessory device, and a remote computing system. The mobile analytical device may be adapted to generate a sensor output that is characteristic of the molecular composition of the sample. The mobile accessory device may be adapted to receive the sensor output from the mobile analytical device. The remote computing system may be adapted to analyze analytical data using AI and/or M-L to make an authentication determination of the sensor output relative to a predefined product database. Furthermore, the remote computing system may include a Monetizer (billing) module that calculates a billing output for a given analysis performed by the mobile analytical device, and the billing output may be calculated as a function of at least one of (a) whether the authentication determination is positive or negative, (b) an identity of the sample, and (c) an identity or group of a user who initiates the given analysis.

We also disclose systems for verifying an authenticity of a sample by analyzing data indicative of a molecular composition of the sample, where the system includes a mobile analytical device, a mobile accessory device, and a remote computing system. The mobile analytical device is adapted to analyze the sample to generate signature data that is indicative of the molecular composition of the sample, the mobile analytical device including one or more first memory devices coupled to one or more first processors. The mobile accessory device is adapted to receive the signature data from the mobile analytical device, the mobile accessory device including one or more second memory devices coupled to one or more second processors. The remote computing system is adapted to analyze the signature data relative to an accepted signature data to produce an authentication determination of the sample, the remote computing system including one or more third memory devices coupled to one or more third processors. The mobile accessory device is adapted to upload the signature data to the remote computing system by a communication network, the remote computing system is adapted to download the authentication determination to the mobile accessory device by the communication network, and the remote computing system is further adapted to calculate a billing output for the sample analysis performed by the mobile analytical device, and to download the billing output to the mobile accessory device by the communication network.

We also disclose methods of determining an authenticity of a food sample by analyzing data indicative of a molecular composition of the sample, where the method includes: (a) analyzing the sample with a mobile analytical device that generates measured signature data indicative of the molecular composition of the sample, the mobile analytical device including one or more first memory devices coupled to one or more first processors; (b) transmitting the measured signature data from the mobile analytical device to a mobile accessory device by a first wireless connection, the mobile accessory device including one or more second memory devices coupled to one or more second processors; (c) transmitting the measured signature data from the mobile accessory device to a remote computing system by a second wireless connection, the remote computing system including one or more third memory devices coupled to one or more third processors, the remote computing system adapted to analyze the measured signature data relative to an accepted signature data to produce an authentication determination of the sample, the remote computing system also adapted to calculate a billing output for the sample analysis performed by the mobile analytical device; and (d) transmitting the authentication determination and the billing output from the remote computing system to the mobile accessory device by the second wireless connection.

We also disclose methods of determining an authenticity of a food sample by analyzing data indicative of a molecular composition of the sample, where the method includes: (a) analyzing the sample with a mobile analytical device that generates measured signature data indicative of the molecular composition of the sample, the mobile analytical device including one or more first memory devices coupled to one or more first processors; (b) transmitting the measured signature data from the mobile analytical device to a mobile accessory device by a first wireless connection, the mobile accessory device including one or more second memory devices coupled to one or more second processors; (c) transmitting a product code associated with the sample from the mobile accessory device to a remote computing system by a second wireless connection, the remote computing system including one or more third memory devices coupled to one or more third processors, the remote computing system having an accepted signature database stored in the one or more third memory devices, the accepted signature database including first accepted signature data associated with the product code, and other accepted signature data associated with other products; (d) selecting the first accepted signature data from the accepted signature database using the product code; (e) transmitting the first accepted signature data from the remote computing system to the mobile accessory device; and (f) using the mobile accessory device to analyze the measured signature data relative to the first accepted signature data to produce an authentication determination of the sample.

We disclose numerous related methods, systems, and articles.

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

The inventive articles, systems, and methods are described in further detail with reference to the accompanying drawings, of which:

FIG. 1 is a schematic perspective view of an exemplary mobile accessory device;

FIG. 2 is a schematic view of a setup in which a mobile analytical device is shown proximate a product of interest and in the process of optically sampling such product;

FIG. 3 is a schematic view of an exemplary mobile analytical device which includes a physical button that can be activated by a user to cause the analytical device to analyze a given sample, which activation of the button may also initiate a sequence of additional actions including causing the remote computing system to calculate a billing output for the sample analysis performed by the mobile analytical device;

FIG. 4 is a diagram of one embodiment of a network-based verification system as disclosed herein;

FIG. 5 is a diagram of a managing platform for a company or entity of mobile analytical device providers, owners, and/or managers; and

FIGS. 6A-6B in combination present a flow chart showing an exemplary operational flow that may be executed by software running on digital processors of the disclosed systems.

In the figures, like reference numerals designate like elements.

### DETAILED DESCRIPTION OF ILLUSTRATIVE EMBODIMENTS

We have developed a new family of network-based verification systems with new and useful features, and combinations of features, as described and summarized herein. The use of spectroscopy, or other measurement techniques that are based on the molecular makeup of the sample, and cloud engines (which may be AI-based) for rapid testing of food or other products enables the systems to provide positive market disruption. For example, whereas conventional verification systems may perform 2-3 tests/hour, the disclosed systems may in many cases perform 20-30 tests/hour. Whereas the test cost (including logistics) may be $150-200/test with conventional systems, it may be as low as $1-2/test for the disclosed systems, in 2019 US dollars. Whereas the test machine cost may be $10 k-25 k with conventional systems, it may he as low as $400 with the disclosed systems, in 2019 US dollars. Whereas the test machine weight may be 5-15 kg with conventional systems, it may be as low as 250 g with the disclosed systems. Whereas conventional systems may require a controlled environment of 25 degrees C., the disclosed systems may be suitable for outdoor temperatures ranging from 0 to 40 degrees C. Whereas the time to receive results may be 1-2 weeks with conventional systems, it may in many cases be as low as 1 minute with the disclosed systems. Test sensitivity may be <1% for conventional systems versus 5% for the disclosed systems. The foregoing comparisons are of course generalized and not necessarily applicable to all conventional systems and all of the disclosed systems.

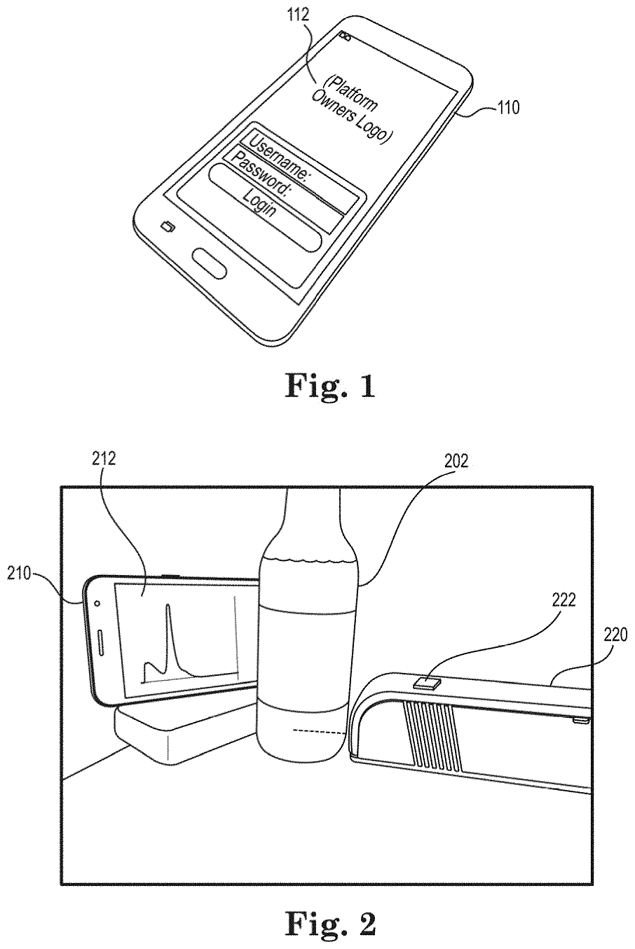

FIG. 1 is a perspective view of an exemplary mobile accessory device 110 that can be used in the disclosed system. In this case, the mobile accessory device 110 is a smart phone into which software has been loaded in the form of an app (application) to permit its use in the disclosed systems. Such a device can communicate and transfer various types of information between a mobile analytical device and a remote computing system, The mobile accessory device 110 may thus for example include a camera such that it can capture an image of a bar code, QR code, or other label information of the sample to be tested so that such labeling information can he uploaded to the remote computing system. The mobile accessory device 110 also preferably includes antenna(s), transmitter(s), receiver(s), and/or other components to provide wireless communication to the mobile analytical device, and to the remote computing system e.g. via a conventional cellular network or other suitable communication network. In FIG. 1, the display/touch screen 112 of the device 110 provides a user interface that can serve the dual purpose of an output function by displaying information to the user, and an input function by allowing the user to enter information or commands by tapping or sliding a finger or stylus on the touch-sensitive surface. As shown, the user can interact with the display/touch screen 112 to login to a predefined system account with a username and password. The mobile accessory device 110 may include alternative or additional input or output devices or components, such as a camera or microphone as input devices, and a speaker as an output device.

FIG. 2 is a schematic view of a setup in which a mobile analytical device 220 is shown proximate a product of interest 202, and in the process of optically sampling and analyzing such product. The mobile analytical device 220 may in some cases be capable of analyzing the product of interest or sample 202 in situ, through the product's outer bottle or container without opening or breaking the seal of the container, as discussed above. In other cases, a mobile analytical device fur use in the disclosed systems may not have such remote sampling capabilities. Exemplary mobile analytical devices suitable for sampling products of interest include the compact spectrometers described in U.S. Pat. No. 10,317,281 (Wang et al.), and analytical devices available from Oak Analytics Inc., Agoura Hills, Calif. The device 220 has a switch or button 222 which the user can press or click to initiate the scan and analysis of the sample 202, as well as other functions including a monetization or billing function as described herein. In the background of the photograph of FIG. 2 is the display screen 212 of a smart phone employed as a mobile accessory device 210, illustrating a typical sensor output in the form of a spectrum (product signature) from a tested sample, the spectrum being indicative of the molecular composition of the sample 202.

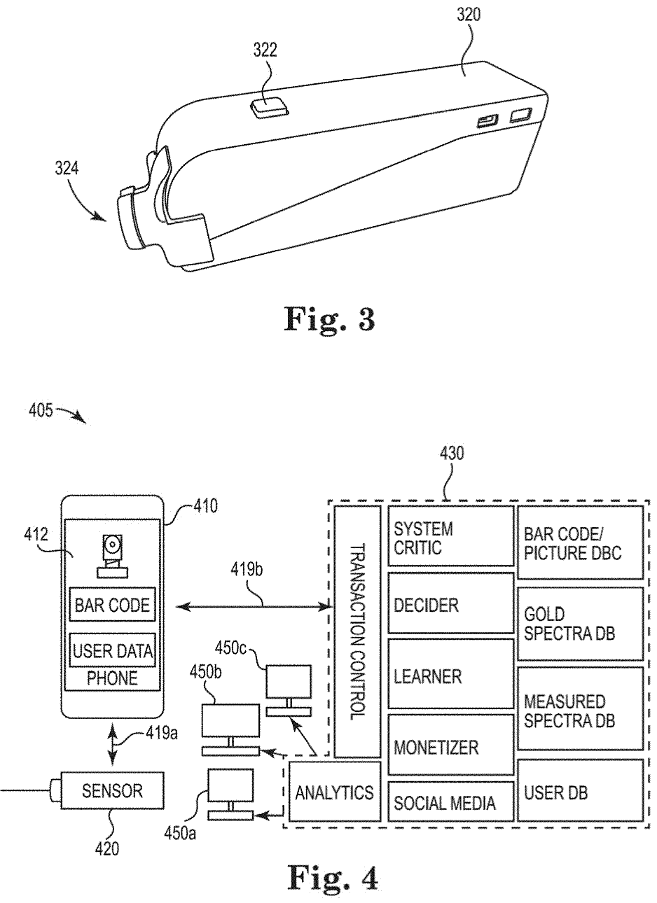

FIG. 3 is a schematic view of an exemplary mobile analytical device 320 which includes a physical button 322 that can be activated or “clicked” by a user to cause the analytical device 320 to analyze a sample of interest, which activation of the button may also initiate a sequence of additional actions including causing the remote computing system to calculate a billing output for the sample analysis performed by the mobile analytical device. Such capability may be referred to as “click, analyze, and bill”, and it represents a vast change from the way in which conventional analytical testing is conducted and invoiced. With the “click, analyze, and bill” capability, the user or their administrator or other designated person or entity can be invoiced within minutes, rather than days or weeks, after performing the analysis on the sample to verify whether it is authentic or not. The “click” here refers to the user pressing or activating the physical button, or other button or switch, provided on the mobile analytical device 320. The verification result itself, i.e. the authentication determination which may be calculated by the remote computing system, may likewise be delivered to the user or other designated person within minutes of activating the button, e.g. by delivering such result to the smart phone or other mobile accessory device being used by the user.

By clicking or otherwise activating the button 322, the customer or user prompts the analytical device, which may be or include a spectral scanner with a testing aperture 324, to initiate a test or measurement, whereupon the analytical device 320 captures a spectral signature of the sample of interest, and sends such signature to a smart phone or other mobile accessory device. The spectral signature, which is indicative of the molecular composition of the sample, may then be transmitted to the cloud (remote network computer(s)), and cloud-based algorithms may be used to make a determination of authenticity, the result of which may then be reported back as a verification result to the initiating user. The system may then bill the customer or user for the scan, thus completing the transaction that was set into motion by the user clicking the button 322.

FIG. 4 is a diagram of one embodiment of a network-based verification system 405 as disclosed herein. Such a system may integrate the Internet of Things (IoT) with AI/M-L, and may be designed to support a large number of transactions per day, e.g., more than 1 million. In FIG. 4, we see in schematic form a mobile analytical device 420 (“Sensor”), a mobile accessory device 410 (“Phone”), and a remote computing system 430. Data may be transmitted in one or both directions between the analytical device 420 and the accessory device 419 via communication link 419a, which may be or include a wireless link such as a BlueTooth™ connection, and/or which may be or include a wired link. Data may be transmitted back and forth between the accessory device 410 and, the computing system 430 via communication link 419b. The link 419b may include the world wide web for internet connectivity in combination with any or all of a wireless connection such as the wireless data connection of a cellular network, or a wireless WiFi connection, or a wired connection.

The mobile accessory device 410 may be the same as or similar to the mobile accessory devices 110, 210 discussed above. The accessory device 410 thus has a display/touch screen 412 that can serve the dual purpose of an output function by displaying information to the user, and an input function by allowing the user to enter information or commands via the touch-sensitive surface. The accessory device 410 may also include other input and output devices. The accessory device 410 also includes one or more suitable digital electronic memory devices configured to store software instructions needed to carry out the functionality described herein, and also configured to store measured sensor data and other digital electronic information. Furthermore, the accessory device 410 includes one or more suitable digital electronic processors coupled to the memory device(s) to carry out its described software functionality.

The mobile analytical device 420 may be the same as or similar to the mobile analytical devices 220, 320 discussed above. The mobile analytical device 420 may be based on Raman spectroscopy or another optical or non-optical measurement technology, but is preferably adapted to generate a sensor output that is characteristic of the molecular composition of the sample. The analytical device preferably has a mechanical switch or button which the user can press or click to initiate the scan and analysis of the sample of interest, as well as other functions as described herein. The analytical device 420 may include one or more suitable digital electronic memory devices configured to store software instructions needed to carry out the functionality described herein, and also configured to store measured sensor data and other digital electronic information, The analytical device 420 may also include one or more suitable digital electronic processors to carry out its described software functionality.

The remote computing system 430 desirably provides a central depository to collect sensor data from measurements taken by users who may be located anywhere in the world, and to provide such global users with reliable information—such as parametrics of their products, parametrics of their analytical devices, and the like—necessary to carry out the product authentication tests and other actions described herein. These tests and actions can be accomplished by any such user with the benefit of the most up-to-date information and parametrics by simply updating the databases and other information in the central remote computing system 430. The updating may take place on a defined maintenance schedule, or on an ongoing real-time basis as information from products and scans of such products is continually being received by the computing system 430. Each user that communicates with the remote computing system 430, regardless of where they are located, may thus perform the most accurate and reliable authentication tests.

The remote computing system 430 is shown as having distinct functions which may be carried out by corresponding software modules, the functions including a Transaction Control function, a System Critic function, a Decider function, a Learner function, a Monetizer function, a Social Media function, an Analytics function, a Bar Code/picture DBC function, a Gold Spectra DB function, a Measured Spectra DB function, and a User DB function. “DB” in this regard refers to a database used for the stated purpose. Each software module may be encoded as digital instructions or other digital information stored in one or more suitable digital electronic memory devices of the remote computing system, and executed by one or more suitable digital electronic processors included in the remote computing system 430.

The Transaction Control function of the remote computing system 430 manages and records data traffic for each transaction as data is fed into the system 430 and various outputs are generated. The Transaction Control function may provide live reports and/or periodic reports to the user or a designated entity or account.

The System Critic function of the remote computing system 430 monitors the health of (diagnostics for) the analytical device 420, as well as user issues and incorrect/exception results.

The Decider function of the remote computing system 430 is the software module that contains one or more decision engine algorithms, i.e., algorithm(s) that receive as inputs at least the raw or adjusted measured signature data from the analytical device on the one hand, and accepted signature data for the product being tested on the other hand, and perform mathematical operations on those data sets to provide a verification result as a determination of authenticity. The algorithms in this software module may employ known mathematical functions and operators.

The Learner function of the remote computing system 430 is the software module that contains machine learning (M-L) algorithms. The M-L algorithms may be used to update a Gold Spectra database as described further below.

The Monetizer, or Monetization, function of the remote computing system 430 is the software module that contains the Billing engine and a Data Analytics Toolkit. The Billing engine may be a conventional billing software. The data collected by the computing system 430 may be monetized in terms of product quality, comparisons, and batch-to-batch variations.

The Social Media function of the remote computing system 430 monitors and scans social media for specific events, and correlates them with test results provided by the verification system 405. Furthermore, the Social Media function may send alerts and/or re-transmissions to social media.

The remote computing system 430 may also include a number of database functions. Each such database function may be or include a database of digital information stored in one car more digital electronic memory devices of the remote computing system.

The Bar Code/picture DBC function of the remote computing system 430 is a database of linear bar codes, 2-dimensional bar codes (QR codes), and/or images of specific products that the verification system 405 has been configured, and programmed, to authenticate. This database may be used to allow the software to recognize and identify the specific product that the user is measuring/scanning and attempting to authenticate.

The Gold Spectra DB function of the remote computing system 430 is a database of accepted signature data for each product that the verification system 405 is capable of authenticating. The accepted signature data, referred to as a Golden signature, for a given product may be an average, or envelope, of numerous scans or measurements made on numerous batches of the product obtained directly from the product's manufacturer, to ensure authenticity of the product and accuracy of the Golden signature data. The Golden signature may be stored in electronic memory in a format that is the same as or similar to the data format used by the mobile analytical device 420 to facilitate comparison between the accepted signature data and the measured sample signature data. The Golden signature may thus be based on an envelope of acceptability that is created using a large number of measurement trials (scans) of authentic product samples, and chemical laboratory verification.

The Measured Spectra DB function of the remote computing system 430 is a database of raw/measured signature data (raw scans). This database is preferably populated with raw signature data from numerous different samples (of each of the products that the verification system 405 is capable of authenticating) taken by numerous different mobile analytical devices 420 and numerous different users at numerous different times. Thus, each time any user of the verification system 405 takes a measurement of any sample using any approved or compatible mobile analytical device 420, the raw signature data from that measurement is communicated from the analytical device 420 to the mobile accessory device 410, and thereafter to the remote computing system 430, where it is added to the database of the Measured Spectra DB.

The User DB function of the remote computing system 430 is a database of users that have registered with the verification system 405. Each user may be identified by a unique username, and may logon to the system with their username and a password. The User database may associate any given user with an entity, such as the user's employer or company, where applicable. Some users may register as individuals, with no company affiliation.

The User DB function may also include one or more calibration tables that can be used to adjust for sensor-to-sensor variability of the mobile analytical devices. The verification system 405 is preferably designed to operate with numerous different users who may wish to analyze or authenticate numerous different products using numerous different mobile analytical devices. The mobile analytical devices, even if all or many of them are nominally of the same design, may incorporate components that exhibit part-to-part variability such as optoelectronic detector(s) or emitter(s), or optical components, and thus the mobile analytical devices, though nominally the same, may produce slightly different raw measurement data on any given sample or product. It therefore can be useful to associate with each specific mobile analytical device a set of calibration data that can be used to correct the device's measurement data for such variability so that analytical measurements taken by different mobile analytical devices on the same product or sample—after being corrected or adjusted using the calibration data—are the same, or more nearly the same. A unique identification number or code can be assigned to each mobile analytical device, and then also assigned to the calibration data for that device, so that after the calibration data is stored in the User database, and when the remote computing system 430 receives measurement data such as a product signature that was generated by an analytical device, the software can retrieve the calibration data for that analytical device using the device's unique identification code. The remote computing system 430 may then use the retrieved calibration data to convert the raw product signature data to calibrated (normalized or corrected) product signature data. In alternative embodiments, the calibration table(s) containing the calibration data may be kept in a separate database function of the remote computing system 430 other than the User database function.

The Analytics function or module of the remote computing system 430 may communicate with various users of the system including consumers, regulators, and manufacturers as shown. Information may be conveyed wirelessly such as by the Blue Tooth™ protocol or other wireless protocols between the mobile analytical device and the mobile accessory device, and between the mobile accessory device and the remote computing system.

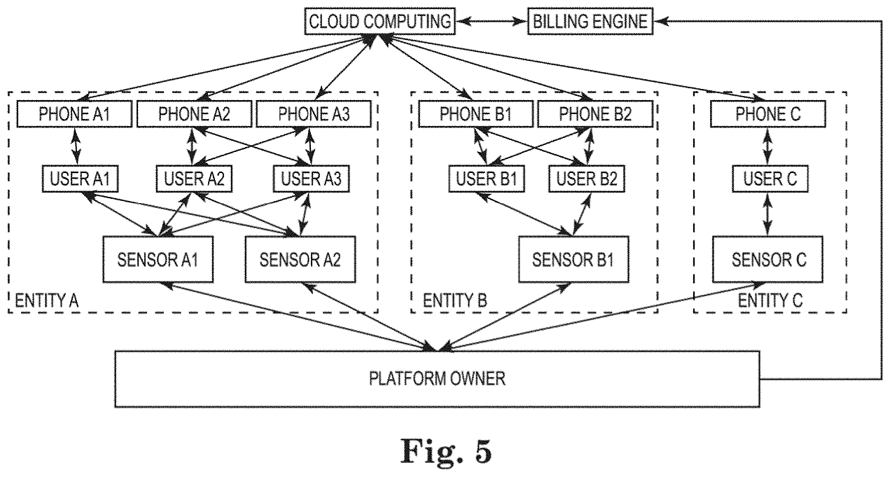

FIG. 5 is a diagram of a managing platform for a company or entity of mobile analytical device providers, owners, and/or managers. As shown, users may be categorized according to group characteristics such as those employed or otherwise associated with entity A versus entity B or entity C. In some cases users in a given group may exchange or use different mobile accessory devices (labeled “Phone” in FIG. 5), and may use different mobile analytical devices (labeled “Sensor” in FIG. 5). In the figure, the entity C may represent an individual account that includes only the one User C, whereas entities A and B may represent companies, organizations, or groups that include multiple unique users.

FIG. 5 may thus represent a managing platform for a company or entity of mobile analytical device providers, owners, and/or managers. The platform owner may perform one, some, or all of the following functions: form a network of users and mobile analytical devices provided by the company or entity: manage users and mobile analytical devices provided by the company or entity; collect and analyze mobile analytical device output data from users; feedback to users judgment or classification, i.e., authentication information such as a verification result, based on the analyzed measured data; and bill users or their respective entities for the service of performing big data analysis. The platform owner may be an analyzer and owner of big data, and can also be a provider, owner, and/or manager of mobile analytical devices.

In some of the many exemplary operational interactions or procedures supported by the system: (1) a user n may connect a mobile accessory device (e.g. Phone) x via Bluetooth™ or other wireless data link with a mobile analytical device (e.g. Sensor) y; (2) user n (account number n) may use the accessory device x to sign into a cloud account n which engages a remote computer n on which the disclosed software is loaded; (3) the cloud or remote computer n checks its database(s) to determine (a) whether user n and mobile analytical device y are on authorized list(s), and (b) whether user n is authorized to use mobile analytical device y; (4) the cloud or remote computer n uses billing engine to automatically bill user n; (5) the cloud or remote computer sends a signal to the mobile accessory device x to authorize use; (6) the user n starts to take one or more measurements or authentication scans of one or more supported products using the mobile analytical device y, and scanned data, i.e., measured product signature data, is transmitted to and stored in the mobile accessory device x; (7) the mobile accessory device x uploads the scanned data to the remote computer n via the interne or cloud; (8) the remote computer or cloud n performs analysis and sends judgment or classification, e.g. verification result(s) corresponding to the tests performed on the product(s) or sample(s), based on analyzed results back to the mobile accessory device x.

A network platform may thus allow its owner to charge a user on a per-use basis of using the cloud big data analysis on the user's uploaded data, wherein the uploaded data are detected locally by the user using a specific proprietary innovative analytical device provided by a company or entity; wherein the big data is established based on the user's uploaded data and the big data is mimed by the network platform owner. In some eases, the network platform owner may also be the provider of the specific proprietary innovative analytical device.

The disclosed systems are configured to automatically perform a sequence of operations in response to the user's single action of activating or clicking a button. The button may be a mechanical or other physical button or switch provided on the mobile analytical device, but in some cases the button may be provided on the mobile accessory device, and may in some cases be a virtual button (e.g., defined as an area on a touch screen). The sequence of operations may preferably include taking an analytical measurement of the product or sample, making an authentication determination based on the measurement and providing the authentication determination (e.g. a verification result) to the user, and generating a bill or invoice and providing that also to the user or to an entity or designated person associated with the user.

For example, upon detecting a click or activation of the button, the software may initiate the following actions:
The mobile analytical device performs an analysis of the sample to produce a product signature (analytical signature data based on molecular characteristics of the sample, the data being for example a string or array of numbers associated with different electromagnetic wavelengths or frequencies sampled by the mobile analytical device), and transmits the product signature to the mobile accessory device. The product signature may in some cases include data on: wavenumber (electromagnetic frequency); measured scattered light intensity at each wavenumber; a measured temperature of the sample; and a measured reference intensity of the laser light or other electromagnetic radiation (if any) that was directed at the sample to take the measurement.
The mobile accessory device may then bundle the product signature with other data about the measurement, such as: a product code, bar code, or other identification information about the tested sample; an identification code for the mobile analytical device; an identification code for the user; the time (including date) the analysis was performed; and the geographic location of the analysis. The time and geographic location may be obtained by on-board capabilities of the mobile accessory device. The mobile accessory device may then transmit this bundled data to the remote computer via the cloud. The Transaction Control module of the remote computer, discussed above, may receive the bundled data from the mobile accessory device.
The remote computer may then perform a number of functions. As discussed above, the remote computer may contain or have access to several databases, including a calibration database or table that can be used to correct for instrument variability from one mobile analytical device to another. Each mobile analytical device authorized for use with the system has a unique identification code, and has been tested against one or more standard samples or materials to generate calibration data that is unique to that device to correct for device-to-device variability. The calibration database, which may be part of the user database described above, contains such calibration data on all mobile analytical devices authorized for use with the system, and is contained in or accessible by the remote computer. The Transaction. Control module of the remote computer may thus access the calibration database, find the calibration data associated with the particular mobile analytical device whose identification code was included in the data bundle, and apply that calibration data to the measured product signature data to produce a calibrated or corrected product signature.
The Transaction Control module of the remote computer may then transmit the calibrated product signature data to the Decider module, discussed above. The Decider module compares the calibrated product signature to the Golden signature and envelope of acceptability associated with the product or sample identification code that was included in the data bundle, in order to calculate a verification result as a measure of the authentication determination, i.e, the degree to which the sample is determined to be authentic based on the analytical measurement. The verification result is determined by mathematical calculations and may initially be a number on a scale from a minimum value to a maximum value, the minimum value corresponding to a fully negative determination (the sample is not authentic due to no or minimal correlation between the measured/calibrated product signature and the Golden signature) and the maximum value corresponding to a fully positive determination (the sample is authentic due to perfect or high correlation between the measured/calibrated product signature and the Golden signature). The verification result can however also be adapted to any user-specified format, for example, a user may want the verification result to be converted to a 6-level code ranging from “no”, to “poor”, to “fair”, to “good”, to “excellent”, to “yes”, or a similar 4-level code, or a 2-level code. The 2-level code may be a simple “yes/no” or “pass/fail” verification result as a simplified version of the original (unfiltered) verification result. In any case, the Decider module may communicate the verification result to the Transaction Control module.
The Transaction Control module of the remote computer may then communicate the verification result to the user's mobile accessory device. The Transaction Control module may also send the calibrated product signature to the Learner module. The Transaction Control module may also send the verification result along with other information relating to the measurement to the Monetizer module.
The Monetizer module may then compute a cost of the measurement based on one or more of: the verification result; the user identification code; the product identification code; the geographic location of the test; and batch-to-batch variations. The computed cost may be sent from the Monetizer module to the Transaction Control module, and from there to the user's mobile accessory device in the form of an invoice or bill.

The verification result may be a factor in computing the cost because a scan or measurement that yields a “pass” result (sample is authentic) may be priced differently than one that yields a “fail” result (sample is not authentic).

The system may bill or invoice the user by creating and updating an electronic invoice for the user that can be accessed at any time by the user, and may be sent electronically and/or physically at the end of the contracted billing cycle. The bill or invoice would typically include who (which user) performed the scan/measurement, the time of the scan, the geographic location of the scan, the identity of the sample, and the verification result achieved. The act of billing or invoicing may be or include: sending a text or other electronic message to the user's phone; generating an electronic document and sending it to the user's email address; generating and printing a paper document and mailing it to the user's physical address; and/or an electronic withdrawal of funds (equal to the calculated cost) from the user's designated bank account, with or without sending the user a message informing them of the withdrawal. In some cases, the act of billing may also include the disclosed system automatically prompting or notifying a third party billing agent, such as the iTunes™ App Store or Google Play™, of the cost, such that the user is contacted by such third party billing agent. The billing by the third party agent, or by the remote computing system 430 or other subsystem of the verification system 405 or its software, may be carried out individually, each time the user executes a click (performs a scan or analysis of the sample), or optionally at an earlier time, e.g. when the user opens a user account or buys a software application (app) that provides them access to the verification system 405.

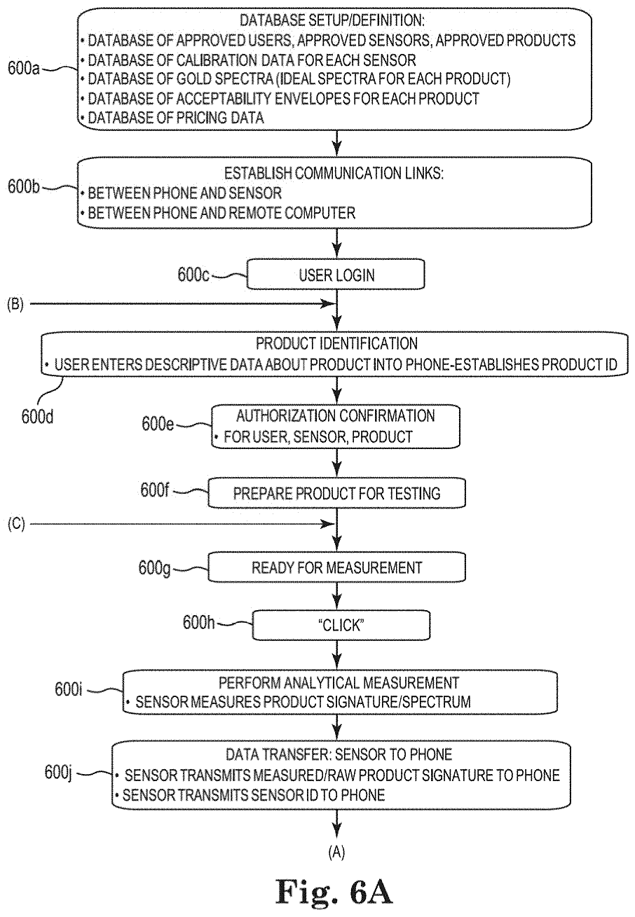

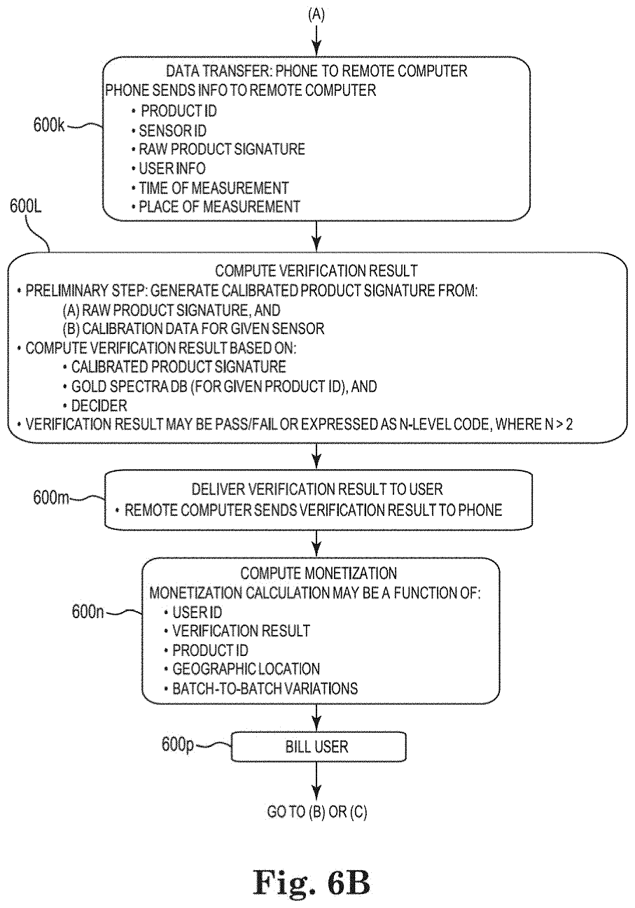

FIGS. 6A-6B provide a flow chart showing an exemplary operational flow that may be executed by the disclosed systems, some steps or aspects of which may be the same as or similar to those already described above. The steps set forth in the flow chart may be carried out by software that is resident on memory device(s) in components or subsystems such as the mobile analytical device 420, the mobile accessory device 410, and/or the remote computing system 430 of the verification system 405 discussed above, such software executed by digital electronic processor(s) resident on such respective components or subsystems.

At step 600a, databases to be used or accessed by the software are set up or otherwise defined. These may include database(s) of approved users, database(s) of approved mobile analytical devices (referred to as sensors in FIGS. 6A-6B), database(s) of approved products capable of being authenticated, database(s) of calibration data for each mobile analytical device, database(s) of Gold spectra (Golden signature data) for each authorized product, database(s) of acceptability envelopes for each authorized product, and database(s) of pricing data, Two or more of these databases may be combined into a single larger database.

At step 600b, communication links are established between the mobile accessory device (referred to as a phone in FIGS. 6A-6B) and the mobile analytical device, and between the mobile accessory device and the remote computer (cloud).

At step 600c, the user logs into an account to set up an active session with a remote computer such as the remote computing system 430. The user may for example enter a username and password using a mobile accessory device such as device 410.

At step 600d, the user enters descriptive data about the product into the mobile accessory device so as to identify to the system which product is to be authenticated. The product descriptive data may in some cases be entered by simply taking a picture of the product's bar code, QR code, or other code or information on the packaging or label of the product, and uploading the picture to the remote computer. In other cases it may involve entering alphanumeric data on a virtual keyboard or other input device of the mobile accessory device.

At step 600e, the system confirms that the particular user, the particular mobile analytical device, and the particular product to be authenticated, axe authorized. This may involve both checking to conform that the codes corresponding to the user, analytical device, and product are all individually recognized and present in the system's database(s), and also checking to confirm that the combination is also authorized. For example, in sonic cases the system's database(s) may authorize a given user to use only certain mobile analytical devices, or to test only certain types of products. Similarly, the system's database(s) may authorize a given type of mobile analytical device to test only certain types of products.

At step 600f, the product is prepared for testing, This may involve the user moving the product or sample into position next to an emitting aperture of the mobile analytical device.

Step 600g indicates the system is live but idle, and ready to take a measurement in response to a command from the user. Step 600h represents such a command being given by the user, in the form of clicking, pressing, or otherwise activating the button provided on the mobile analytical device, the command referred to as a “click”. As noted elsewhere, the click may take other forms, such as pressing or activating a virtual button or other button or switch on the user's mobile accessory device. Alternatively, the click may be accomplished by other input mechanisms, including non-tactile mechanisms, such as voice activation from a microphone on the mobile accessory device or on the mobile analytical device.

In response to the click command input, the system performs a sequence of steps including one, some, or all of steps 600i through 600p.

At step 600i, the mobile analytical device measures the product signature data indicative of molecular characteristics of the sample. The data may be obtained by Raman spectroscopy or other suitable analytical measurement techniques performed by the mobile analytical device. At step 600j, the (raw) product signature data is transmitted to the mobile accessory device. The mobile analytical device may also at this time transmit its unique sensor identification code to the mobile accessory device, or that transmission may occur at an earlier time such as at step 600b.

Thereafter (follow reference number (A) to FIG. 6B), in step 600k, the mobile accessory device may bundle and transmit some or all of the following data, which relates to the measurement that was performed in the previous step 600j, to the remote computer: the identification code for the product or sample; the identification code for the mobile analytical device; the (raw) product signature data taken from the sample; the identification code for the user; the current time, which may include both the date and the local time in hours and minutes as defined in the mobile accessory device; and the place of the measurement, e.g. in GPS coordinates as defined in the mobile accessory device.

The system then proceeds with computing the verification result in step 600L. In a preliminary step, the system may compute an adjusted or calibrated product signature based on the (raw) product signature data and calibration data for the mobile analytical device (obtained from the system's calibration database using the device's identification code). The calibrated product signature is then transmitted to the Decider module, which computes the verification result by comparing the calibrated product signature to its corresponding Gold spectra (Golden signature data) for the tested product. Acceptability envelope data may also be used in these calculations. The verification result may be expressed as an integer or floating point number between a maximum and minimum limit, or as a pass fail or yes/no indicator, or as an n-level code, such as a 4-level code, or a 6-level code. The desired format of the verification result may be a setting in the profile of the user or the user's company, group, or entity.

At step 600m, the verification result is transmitted from the remote computer to the user, e.g. by transmitting the verification result to the mobile accessory device, and displaying the result on the display screen of that device. The verification result may also be stored in an electronic log or account of the user or the user's entity.

At step 600n, the Monetizer module of the system, or other aspect of the system software, calculates a number corresponding to a cost assessed by the platform owner for the testing and analysis services that were just provided to the user. The calculated cost of the measurement may be based on one or more of: the user identification code; the verification result; the product identification code; the geographic location of the test; and batch-to-batch variations.

At step 600p, the user may be billed for the analysis by transmitting an invoice containing the computed cost to the user. The billed cost may be a function of several factors as described above. After billing the user, software control may return to point (B), to prompt the user to identify another product to be analyzed, or to point (C), to prompt the user to re-measure the same product/sample, or to measure a different sample of the same type of product, e.g., a different second bottle of the same product type.

The system may be configured to distinguish between a successful measurement that yields a verification result of “fail”, and a failed or erroneous measurement. In the former case, the mobile analytical device may detect an adequate backscatter signal level from the sample, but when that backscattered signal (product signature) is analyzed, its correlation with the Golden signature is negative, i.e., not authentic. In the latter case, the mobile analytical device may detect little or no backscatter signal level at all, making it difficult or impossible to make any authentication calculation. The latter situation may occur if the user fails to position the product or sample up against the measurement aperture of the analytical device, resulting in an “inadequate signal” error or “sample not present” error. When such an error is detected, the as software may immediately shut off the mobile analytical device.

In response to an otherwise successful measurement that yields a negative or “fail” result for authenticity, the system may prompt the user to take remedial steps to re-check the measurement, such as running a calibration scan and then running a second scan of the product/sample. The system may treat these additional remedial steps as being included in the assessed cost of the “fail” authenticity assessment.

In alternative embodiments to the arrangement shown in FIG. 4, a portion of the Decider module's function, and other functionality, may be moved from the remote computing system 430 to the mobile accessory device 410. One reason for doing this may be in cases where the user's internet service is sporadic, unreliable, or otherwise not always available. In such cases, a dead or “down” communication link 419b between the mobile accessory device 410 and the remote computing system 430 may cause the user to experience long delays while waiting for the product signature data and related data to be sent to the remote computing system 430, or waiting for the verification result, as computed by the remote computing system 430, to be sent to the mobile accessory device 410. To avoid this, some of the functionality of the remote computing system 430 can be transferred to, or replicated in, the mobile accessory device 410 to allow the mobile accessory device 410 to calculate the verification result itself, and then later communicate the result to the remote computer as part of a syncing operation. To accomplish this, the particular calibration data for the mobile analytical device being used in the current authentication test is located (found and identified) in the calibration database of the remote computing system 410, and transmitted to the mobile accessory device. The particular Gold spectra (Golden signature data) for the product to be authenticated is similarly located (found and identified) in the Gold Spectra database of the remote computing system 410, and also transmitted to the mobile accessory device. Equations, decision rules, scanner profiles, and other computational protocols necessary for the mobile accessory device to calculate a calibrated product signature from the raw product signature and the calibration data, and to calculate a verification result from the calibrated product signature and the Golden signature data, are also downloaded to or otherwise placed in the digital electronic memory device(s) of the mobile accessory device 410. The transfer of the calibration data and the Golden signature data, and optionally the other information, may be conveniently accomplished at the time the user logs onto the system (see step 600c above), when the internet connection is “up” and the communication link 419b is active or live.

The product authentication systems described herein, and components thereof, may utilize the technologies known as Artificial Intelligence (AI), Machine-Learning (M-L), Internet of Things (IoT), and Big Data. In other cases the disclosed systems may use only some, or even no such technologies.

For example, IoT is a system of interrelated computing devices, mechanical and digital machines that are provided with unique identifiers (UIDs) and the ability to transfer data over a network with human-to-human or human-to-computer interaction. Any of the mobile analytical devices 220, 320, 420 disclosed herein may be configured as an IoT device, and as such, the associated mobile accessory device 110, 210, 410 may be entirely omitted from the authentication system by moving functionality from the accessory device to the analytical device. Furthermore, the mobile analytical device as an IoT device may also be given the calibration capabilities and decision capabilities discussed above in connection with the sporadic internet issue.

AI or M-L may also be employed in the disclosed systems. In one application, the system software can be configured to continually update the Gold Spectra database discussed above in response to authentication determinations that are positive, e.g., whose verification result is computed as a “pass” on a pass/fail scale, or that is above a specified threshold value between the minimum and maximum limits. When such a result is obtained from a user's analysis of a given product, the system may take the specific product signature data, or the specific calibrated product signature data, that produced the positive authentication determination, and use that data to revise the Golden signature data for the given product (which Golden signature data is a small portion of the Gold Spectra database, which contains signature data on numerous different products). The specific (calibrated) product signature data may for example be combined with preexisting Golden signature data in a type of weighted averaging computation. In this way, Golden signature data in the Gold Spectra database may be continually updated substantially in real time, using only product signature data associated with a positive authentication determination, such product signature data potentially obtained by users located throughout the world using different mobile analytical devices.

In an alternative arrangement, the Gold Spectra database can be updated as a batch process on a periodic basis or other regular basis. In this case, product signatures (multiple sets of product signature data), or calibrated product signatures, that yielded positive authentication results over a defined period of time for a given product, can be used to revise the preexisting Gold signature data for that product in a similar way, and this can be done for all products represented in the Gold Spectra database.

Big Data may also be employed in the disclosed systems.

Unless otherwise indicated, all numbers expressing quantities, measured properties, and so forth used in the specification and claims are to be understood as being modified by the term “about”. Accordingly, unless indicated to the contrary, the numerical parameters set forth in the specification and claims are approximations that can vary depending on the desired properties sought to be obtained by those skilled in the art utilizing the teachings of the present application. Not to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, to the extent any numerical values are set forth in specific examples described herein, they are reported as precisely as reasonably possible. Any numerical value, however, may well contain errors associated with testing or measurement limitations.

Various modifications and alterations of this invention will be apparent to those skilled in the art without departing from the spirit and scope of this invention, which is not limited to the illustrative embodiments set forth herein. The reader should assume that features of one disclosed embodiment can also be applied to all other disclosed embodiments unless otherwise indicated. All U.S. patents, patent application publications, and other patent and non-patent documents referred to herein are incorporated by reference, to the extent they do not contradict the foregoing disclosure.

What is claimed is:

1. A system for verifying an authenticity of a sample by analyzing data indicative of a molecular composition of the sample, the system comprising:
a mobile analytical device adapted to analyze the sample to generate signature data that is indicative of the molecular composition of the sample, the mobile analytical device including one or more first memory devices coupled to one or more first processors;
a mobile accessory device adapted to receive the signature data from the mobile analytical device, the mobile accessory device including one or more second memory devices coupled to one or more second processors; and
a remote computing system adapted to analyze the signature data relative to an accepted signature data to produce an authentication determination of the sample, the remote computing system including one or more third memory devices coupled to one or more third processors;
wherein the mobile accessory device is adapted to upload the signature data to the remote computing system by a communication network;
wherein the remote computing system is adapted to download the authentication determination to the mobile accessory device by the communication network; and
wherein the remote computing system is further adapted to calculate a billing output for the sample analysis performed by the mobile analytical device, and to download the billing output to the mobile accessory device by the communication network.

2. The system of claim 2, wherein the mobile analytical device includes a button which, when activated by a user, causes the mobile analytical device to analyze the sample, generate the signature data, and transmit the signature data to the mobile accessory device, and further causes the remote computing system to make the authentication determination, download the authentication determination to the mobile accessory device, and download the billing output to the mobile accessory device.

3. The system of claim 1, wherein the mobile accessory device includes a smart phone.

4. The system of claim 1, wherein the mobile analytical device includes a compact spectrometer, and wherein the signature data includes a Raman spectrum.

5. The system of claim 1, wherein the remote computing system is a cloud-based computing system.

6. The system of claim 1, wherein the billing output is calculated as a function of the authentication determination.

7. The system of claim 1, wherein the billing output is calculated as a function of an identity of the sample.

8. The system of claim 7, wherein the billing output is calculated as a function of an identity or group of the user.

9. A method of determining an authenticity of a food sample by analyzing data indicative of a molecular composition of the sample, comprising:
(a) analyzing the sample with a mobile analytical device that generates measured signature data indicative of the molecular composition of the sample, the mobile analytical device including one or more first memory devices coupled to one or more first processors;
(b) transmitting the measured signature data from the mobile analytical device to a mobile accessory device by a first wireless connection, the mobile accessory device including one or more second memory devices coupled to one or more second processors;
(c) transmitting the measured signature data from the mobile accessory device to a remote computing system by a second wireless connection, the remote computing system including one or more third memory devices coupled to one or more third processors, the remote computing system adapted to analyze the measured signature data relative to an accepted signature data to produce an authentication determination of the sample, the remote computing system also adapted to calculate a billing output for the sample analysis performed by the mobile analytical device; and
(d) transmitting the authentication determination and the billing output from the remote computing system to the mobile accessory device by the second wireless connection.

10. The method of claim 9, wherein a button is included on the mobile analytical device or on the mobile accessory device, and wherein steps (a), (b), (c), and (d) are triggered by a user activating the button.

11. The method of claim 9, wherein the mobile analytical device has a first unique identification code stored in the one or more first memory devices, wherein the remote computing system has a calibration database stored in the one or more third memory devices, the calibration database including first calibration data on the mobile analytical device and other calibration data on other mobile analytical devices, and wherein the method further comprises:
transmitting the first unique identification code from the mobile analytical device to the mobile accessory device, and from the mobile accessory device to the remote computing system;
selecting the first calibration data from the calibration database using the first unique identification code; and
producing a calibrated signature data from the measured signature data using the first calibration data.

12. The method of claim 11, wherein the remote computing device produces the authentication determination by comparing the calibrated signature data to the accepted signature data.

13. The method of claim 9, wherein the sample is associated with a first product, wherein the remote computing device has an accepted signature database stored in the one or more third memory devices, the accepted signature database including the accepted signature data associated with the first product and other accepted signature data associated with other products, and wherein the method further comprises:
transmitting a first product code associated with fire sample from the mobile accessory device to the remote computing system; and
selecting the accepted signature data from the accepted signature database using the first product code.

14. The method of claim 13, further comprising:
updating the accepted signature database in response to the authentication determination being positive.

15. The method of claim 14, wherein the remote computing device produces a calibrated signature data from the measured signal data, and wherein the accepted signature database is updated using the calibrated signature data.

16. A method of determining an authenticity of a food sample by analyzing data indicative of a molecular composition of the sample, comprising:
(a) analyzing the sample with a mobile analytical device that generates measured signature data indicative of the molecular composition of the sample, the mobile analytical device including one or more first memory devices coupled to one or more first processors;
(b) transmitting the measured signature data from the mobile analytical device to a mobile accessory device by a first wireless connection, the mobile accessory device including one or more second memory devices coupled to one or more second processors;
(c) transmitting a product code associated with the sample from the mobile accessory device to a remote computing system by a second wireless connection, the remote computing system including one or more third memory devices coupled to one or more third processors, the remote computing system having an accepted signature database stored in the one or more third memory devices, the accepted signature database including first accepted signature data associated with the product code, and other accepted signature data associated with other products;
(d) selecting the first accepted signature data from the accepted signature database using the product code;
(e) transmitting the first accepted signature data from the remote computing system to the mobile accessory device; and
(f) using the mobile accessory device to analyze the measured signature data relative to the first accepted signature data to produce an authentication determination of the sample.

17. The method of claim 16, wherein the mobile analytical device has a first unique identification code stored in the one or more first memory devices, wherein the remote computing system has a calibration database stored in the one or more third memory devices, the calibration database including first calibration data on the mobile analytical device and other calibration data on other mobile analytical devices, and wherein the method further comprises:
transmitting the first unique identification code from the mobile analytical device to the mobile accessory device, and from the mobile accessory device to the remote computing system;
selecting the first calibration data from the calibration database using the first unique identification code; and
transmitting the first calibration data from the remote computing system to the mobile accessory device; and
using the mobile accessory device to produce a calibrated signature data from the measured signature data using the first calibration data.
